(19) United States
(12) Patent Application Publication
(10) Pub. No.: US 2024/0298917 A1
(43) Pub. Date: Sep. 12, 2024

(54) METHOD FOR GENERATING AN IMAGING IMAGE DATASET, MAGNETIC RESONANCE APPARATUS AND COMPUTER PROGRAM PRODUCT

(71) Applicant: Siemens Healthineers AG, Forchheim (DE)

(72) Inventor: Patrick Liebig, Erlangen (DE)

(21) Appl. No.: 18/596,938

(22) Filed: Mar. 6, 2024

(30) Foreign Application Priority Data
Mar. 9, 2023 (CN) 202310257382.5

(51) Int. Cl. A61B 5/055; G06T 5/50; G06T 11/00

(52) U.S. Cl. CPC A61B 5/055; G06T 5/50; G06T 11/008; G06T 2207/20221

(57) ABSTRACT

A method for generating an imaging image dataset includes: acquiring a plurality of prescan raw datasets; generating a plurality of prescan single coil image datasets by reconstructing the one prescan raw dataset in each case; generating a combined prescan image dataset by combining the plurality of prescan single coil image datasets, wherein the generation of the combined prescan image dataset includes an establishment of a plurality of phase values, each of which is associated with an image element of the combined prescan image dataset; acquiring a plurality of imaging raw datasets, each with one of the plurality of coil elements; generating a plurality of imaging single coil image datasets by reconstructing the one imaging raw dataset in each case; and generating a combined imaging image dataset by combining the plurality of imaging single coil image datasets while taking account of the phase values of the combined prescan image dataset.

The present patent document claims the benefit of Chinese Patent Application No. 202310257382.5, filed Mar. 9, 2023, which is hereby incorporated by reference in its entirety.

### TECHNICAL FIELD

The disclosure relates to a method for generating an imaging image dataset, a magnetic resonance apparatus, and a computer program product.

### BACKGROUND

In medical technology, imaging by magnetic resonance (MR), also referred to as magnetic resonance tomography (MRT) or magnetic resonance imaging (MRI) is distinguished by high soft-tissue contrast levels. Herein, an examination object, for example a patient, may be positioned in an examination space of a magnetic resonance apparatus. During a magnetic resonance scan, high frequency (RF) transmitted pulses may be radiated into the examination object, in accordance with a magnetic resonance protocol, with the aid of a high frequency antenna unit of the magnetic resonance apparatus and thus generate a B1 field. Nuclear spins are excited in the examination object by way of the generated transmitted pulses, so that by gradient pulses, spatially encoded magnetic resonance signals are triggered. A magnetic resonance signal may thus be regarded, in particular, as a patient-specific signal response of the transmitted pulse. The magnetic resonance signals are received by a high frequency receiving unit of the magnetic resonance apparatus as raw data and are used to generate image data, in particular magnetic resonance mappings.

In magnetic resonance scans by a magnetic resonance apparatus that has a particularly high, in particular an ultra-high, main magnet field strength (e.g. 7 T or more) (ultra-high field MRI (UHF MRI)), the transmitted pulses may be generated by local coils, in particular surface coils. The local coils may be arranged directly on the examination object, in particular, on the body of the patient to be examined.

In the case of low field strengths, however (e.g., 1.5 or 3 T), the transmitted pulses are mostly generated by a body coil fixedly installed in the magnetic resonance apparatus. This body coil may be situated behind the housing of the bore, in particular a tunnel, in the magnetic resonance apparatus. The body coil is therefore not arranged directly on the examination object, in particular, on the body of the patient to be examined, but rather has a greater separation from the examination object. The body coil may be able to emit transmitted pulses and also to receive magnetic resonance signals. In certain examples, the body coil has a homogeneous reception field according to its size. Local coils, in particular their coil elements, however, may be somewhat smaller and thus do not cover the entire examination object, so that their reception field may not be homogeneous.

In order to generate a magnetic resonance mapping from the raw data that is acquired, in particular received, with the coil elements of the one or more local coils, a single coil image may be reconstructed for each coil element. These single coil images of the different coil elements are then combined into an overall image.

In order to obtain a magnetic resonance mapping of high quality from the combination of the single coil images, conventionally a reference phase information item is used in order to correct a relative phase of the different coil elements; thereby, signal cancellations may advantageously be prevented. This reference phase information item should therein extend over the entire region that is covered by the different coil elements. A reference phase information item of this type is recorded by way of the body coil, which has a large coverage region. However, a magnetic resonance apparatus with a high main magnet field strength may have no such body coil so that also no reference phase information item may be established in this way.

In a magnetic resonance apparatus that has no body coil, but only coil elements of local coils, the data of the coil element that has a maximum signal intensity may be used as a reference. This has the consequence that although the best available coil element is used, this coil element may not irradiate the entire examination object. However, in the regions in which this best available coil element does not receive a signal, the reference phase information item cannot be determined, so that signal cancellations may occur, in particular, in these regions.

Alternatively, according to the publication by Souheil J. Inati, Michael Hansen, Peter Kellman: A Solution to the Phase Problem in Adaptive Coil Combination. Proc. Intl. Soc. Mag. 3eason. Med. 21 (2013) 2672, an adaptive coil combination is applied directly to the imaging raw data. Herein, the data of the coil elements is combined so that the combined phase is flat, such that no reference phase information item is needed. However, this method requires a very large computation effort.

### SUMMARY AND DESCRIPTION

An object of the present disclosure is to carry out a coil combination in a time-efficient manner, even without a reference phase information item of a body coil. The scope of the present disclosure is defined solely by the appended claims and is not affected to any degree by the statements within this summary. The present embodiments may obviate one or more of the drawbacks or limitations in the related art.

A method is disclosed for generating an imaging image dataset based on raw data acquired by a magnetic resonance apparatus. Therein, the magnetic resonance apparatus includes a local coil arrangement with a plurality of coil elements. According to the method, a plurality of prescan raw datasets are acquired, each with one of the plurality of coil elements, i.e., one of the plurality of prescan raw datasets is acquired with each of the plurality of coil elements. For example, a first prescan raw dataset is acquired with a first coil element of the plurality of coil elements, a second prescan raw dataset is acquired with a second coil element of the plurality of coil elements, etc.

Furthermore, a plurality of prescan single coil image datasets are generated, in that the prescan raw datasets are reconstructed separately from one another. In particular, exactly one of the plurality of prescan single coil image datasets is associated with each of the plurality of coil elements. For example, a first prescan single coil image dataset is reconstructed from the first prescan raw dataset, a second prescan single coil image dataset is reconstructed from the second prescan raw dataset, etc.

Furthermore, a combined prescan image dataset is generated by a combination of the plurality of prescan single coil image datasets. For example, the first prescan single coil image dataset, the second prescan single coil image dataset, etc., are combined to form the prescan image dataset. Therein, the generation of the combined prescan image dataset includes an establishment of a plurality of phase values, each of which is associated with an image element of the combined prescan image dataset. Therein, the plurality of phase values of the combined prescan image dataset are established in that the plurality of phase values of the combined prescan image dataset have no phase jump, in particular no phase jump of 360° (in degree measure) or 2π (in radian measure). A phase jump exists, in particular, if a phase value changes abruptly. A phase jump exists, in particular, if a phase value of an image element differs significantly from a phase value of an adjoining image element. A phase jump of this kind may also be referred to as a singularity.

Furthermore, a plurality of imaging raw datasets is acquired, each acquired with one of the plurality of coil elements, i.e., one of the plurality of imaging raw datasets is acquired with each of the plurality of coil elements. For example, a first imaging raw dataset is acquired with the first coil element of the plurality of coil elements, a second imaging raw dataset is acquired with a second coil element of the plurality of coil elements, etc.

Furthermore, a plurality of imaging single coil image datasets is generated, in that the imaging raw datasets are, in particular, reconstructed separately from one another. For example, a first imaging single coil image dataset is reconstructed from the first imaging raw dataset, a second imaging single coil image dataset is reconstructed from the second imaging raw dataset, etc.

Furthermore, a combined imaging image dataset is generated by a combination of the plurality of imaging single coil image datasets while taking account of the phase values of the combined prescan image dataset.

For example, in a SENSE-based (SENSE: SENSitivity Encoding) reconstruction, the phase of coil sensitivities of the plurality of coil elements is corrected and the combined image is generated in the k-space-to-image reconstruction.

For example, in a GRAPPA-based (GRAPPA: GenerRalized Autocalibrating Partially Parallel Acquisition) reconstruction, the single coil images are generated and subsequently joined together into a combined image by the reference phase. In particular, the phases of the plurality of imaging single coil image datasets are corrected in the image space.

The acquisition of the plurality of prescan raw datasets and the acquisition of the plurality of imaging raw datasets may take place in the context of an execution of a magnetic resonance protocol. Such a magnetic resonance protocol may include a magnetic resonance sequence for the acquisition of the plurality of prescan raw datasets and a magnetic resonance sequence for the acquisition of the plurality of imaging raw datasets. For example, the prescan raw datasets are recorded before the imaging raw datasets. The plurality of prescan raw datasets may not be used directly for generating magnetic resonance mappings, but, for example, for calibrating and/or setting any subsequent imaging magnetic resonance sequences.

The magnetic resonance apparatus may have a main magnet field of more than 5 T and/or no body coil fixedly installed in the magnetic resonance apparatus.

The local coil arrangement may include one or more local coils. In particular, the local coil arrangement may include one or more local coils. A local coil may include at least one coil element. A coil element may include an antenna that is configured to receive an HF signal, in particular a magnetic resonance signal. The coil element may have a loop form. The plurality of coil elements of the local coil arrangement may form a phased array arrangement. Each of the plurality of coil elements may be part of a receiving channel and/or a transmitting channel of the magnetic resonance apparatus.

The acquisition of the raw datasets, in particular the prescan raw datasets and/or the imaging raw datasets, may include receiving of magnetic resonance signals. The raw datasets may be a (digitized) form of the magnetic resonance signals. The raw datasets may describe raw data in a k-space.

To be differentiated therefrom are the image datasets, e.g., the prescan single coil image datasets, the prescan image datasets, the imaging single coil image datasets, and/or the combined imaging image dataset. An image dataset may include image data. The image datasets may be a (digitized) form of one or more magnetic resonance mappings. The image datasets may describe image data in an image space. The raw data in the k-space may be transformed, for example, by a two-dimensional or three-dimensional Fourier transform into image data in the image space.

Each image dataset may have a large number of individual image elements (e.g. pixels or voxels), in particular image points. An image element may be described by way of coordinates of the image space. In particular, an amplitude value and/or a phase value is associated with each image element of an image dataset. The amplitude value and the phase value may be regarded as components of a complex-valued information item which is associated with an image element. The amplitude values associated with the image elements and/or a phase value may be understood as image data. The amplitude values may each describe a grayscale step of an associated image element of a magnetic resonance mapping.

The k-space may be a two or three-dimensional data model including (digitized) raw data. In particular, the k-space is identical with a raw data matrix. The raw data matrix is filled, for example by conventional measuring methods, with rows. The axes of the k-space are identified, for example as kₓ (horizontal axis of the k-space) and k_{y} (vertical axis of the k-space). The data points of the plane lying within these axes represent the spatial frequencies. By a Fourier transform, this spatial frequency data may be converted into image data (that is to be displayed).

The image-space may be a two or three-dimensional data model including (digitized) image data. The axes of the image-space are identified, for example, as x (horizontal axis of the image space) and y (vertical axis of the image space). In particular, the image space is identical with an image matrix.

A magnetic resonance mapping may have a large number of individual pixels. The pixels are arranged, for example, chessboard-like to the image matrix. Each pixel in the image matrix may have a particular grayscale value that corresponds, in particular, to the amplitude value of the pixel. Considered together, this grayscale matrix results in a pictorial representation of the magnetic resonance mapping.

Each point in the raw data matrix may contain partial information of the overall image. A point in the image matrix therefore certainly does not represent a point in the raw data matrix. The central region of the raw data matrix may determine the coarse structure and the contrast in the magnetic resonance mapping. The outer region of the raw data matrix may supply information regarding edges and outlines in the magnetic resonance mapping, finer structures, and finally also determine the resolution.

A reconstruction of a raw dataset, in particular a prescan raw dataset and/or an imaging raw dataset may (as already described), include a Fourier transform, in particular a fast Fourier transform (FFT). However, it is also conceivable to reconstruct an image dataset from a raw dataset by other methods, for example, by a neural network and/or a trained function.

The combined prescan image dataset may describe a reference phase that is suitable for describing a phase relationship between imaging single coil image data of the imaging single coil image datasets.

The imaging single coil image data of the imaging single coil image datasets have, in particular, phase values that are each associated with an image element. With the aid of the reference phase, the phase values of the imaging single coil image datasets may advantageously be corrected during the generating of the combined imaging image dataset (relative to one another).

Each prescan raw dataset of the plurality of prescan raw datasets may have a smaller resolution than the corresponding imaging raw datasets. The plurality of prescan raw datasets may have a lower number of items of raw data than the corresponding imaging raw datasets. The prescan raw datasets and the imaging raw datasets that have been acquired with the same coil element may correspond to one another.

Advantageously, due to their lower resolution and/or number of items of raw data, the prescan raw datasets may be recorded particularly quickly. Advantageously, the prescan raw datasets serve at least one further purpose in the magnetic resonance scan, in particular during the acquisition and/or processing of imaging raw data. For example, on the basis of the prescan raw datasets, an acquisition region (for example, a slice and/or a volume) of the plurality of imaging raw datasets may be stipulated. In particular, the magnitudes of datasets that have been acquired by a magnetic resonance apparatus with a main magnet field strength of, for example, 1.5 T or 3 T, are advantageously corrected on the basis of the prescan raw datasets.

It is, however, possible in principle that the prescan raw datasets have a higher resolution than the corresponding imaging raw datasets. However, the necessary computing time may increase quadratically with the resolution.

The method may further include an acquisition of a plurality of further imaging raw datasets, each with one of the plurality of coil elements, generating a plurality of further imaging single coil image datasets by reconstruction of the respective one further imaging raw dataset and generating a further combined imaging image dataset by a combination of the plurality of further imaging single coil image datasets, while taking account of the phase values of the combined prescan image dataset.

The phase values of the combined prescan image dataset may be used as a reference phase not only for a combination of a single imaging image dataset, but for a combination of one of a plurality of imaging image datasets. Advantageously thereby, a generation of a plurality of combined imaging image datasets may be carried out particularly quickly, since the phase values are established only once on the basis of the prescan raw datasets.

An embodiment of the method for generating the imaging image dataset provides that each of the plurality of prescan single coil image datasets includes a plurality of phase values which are each associated with one image element of the respective prescan single coil image dataset. The plurality of phase values of the combined prescan image dataset, which are each associated with one image element of the combined prescan image dataset, are established while taking account of a plurality of phase values of the plurality of prescan single coil image datasets. Therein, the phase values of the plurality of prescan single coil image datasets are associated with image elements arranged in a predetermined, in particular spatial, region round the respective image element of the combined prescan image dataset.

Advantageously, it may be achieved by taking account of the phase values in the predetermined region, that the plurality of phase values of the combined prescan image dataset have no singularity, in particular, no phase jump.

The image element may be described by a point in the image space. For example, a point of this type is specified by (spatial) coordinates, e.g., an x-coordinate and a y-coordinate (in the case of a two-dimensional image dataset) or an x-coordinate, a y-coordinate, and a z-coordinate (in the case of a three-dimensional image dataset).

For example, the image element of the combined prescan image dataset is described by the coordinates x=x₀=0 and y=y₀=0. For example, the predetermined region is specified by x=−p, . . . , 0, . . . , p and y=−q, . . . , 0, . . . , q, where p and q are natural numbers. (The x-region from −p to p may also be denoted, in particular, as Δx; the y-region from −q to q may also be denoted, in particular, as Δy.) Thus, for the x-axis, there results a total of 2p+1=N image element coordinates and for the y-axis, a total of 2q+1=M image element coordinates. Thus, for the phase value of the combined prescan Image dataset, the result is that a predetermined region which includes N×M phase values of the plurality of prescan single coil image datasets is taken into account.

The N×M phase values may be represented as a matrix with N×M matrix elements. The rows of such a matrix then correspond, for example, to the x-axis with an x-coordinate for each matrix element. The columns of such a matrix then correspond, for example, to the y-axis with a y-coordinate for each matrix element. In certain examples, N=M, i.e., the predetermined region around the respective image element is square.

A possible embodiment provides that for determining a phase value associated with an image element, a matrix is generated that includes the phase values of all the prescan single coil image datasets associated with the image elements situated in the predetermined region and eigenvalues of the generated matrix are established. The phase value of the combined prescan image dataset associated with the image element is stipulated on the basis of the maximum established eigenvalue.

For example, L prescan single coil image datasets are available that have been generated from L prescan single coil image datasets, which themselves have been acquired from L coil elements. In order to establish a phase value of an image element, for example, for each of the L prescan single coil image datasets, a column of the matrix to be generated is established that includes N×M phase values, each of which is associated with a matrix element of the column. The matrix to be generated thus includes L×(N×M) matrix elements. From this L×(N×M) matrix, for example, K eigenvalues are determined by known mathematical methods, from which in turn the maximum eigenvalue is determined. On the basis of this maximum eigenvalue, the phase value for the associated image element is then established. In particular, the phase of the eigenvalue corresponds to the phase value that is to be determined for the associated image element.

If, for example, as a predetermined region, a square region of 7×7 phase values is selected, each column includes 7×7=49 matrix elements, each having one phase value. If, for example, 32 prescan single coil image datasets are available, the result for the dimension of the matrix to be generated is 32×49, i.e., the matrix to be generated would be a 32×49 matrix, from which the maximum eigenvalues are established.

Based on the phase values of the combined prescan image dataset, coil combination weights may be established, on the basis of which the plurality of imaging single coil image datasets are combined to the combined imaging image dataset. In order to establish the coil combination weights, various methods are known in the prior art. For example, for this purpose, splines are fitted to the single coil images or the ESPIRiT method (Uecker et al., ESPIRiT—an eigenvalue approach to autocalibrating parallel MRI: where SENSE meets GRAPPA. Magn Reson Med. 2014 March; 71(3):990-1001) is used.

Furthermore, a magnetic resonance apparatus is proposed that is configured to carry out a method as described above. The advantages of the magnetic resonance apparatus may correspond to the advantages of the method for generating a combined imaging image dataset that have been described in detail above. Features, advantages, or alternative embodiments mentioned herein may also be transferred to the other claimed subject matter and vice versa.

Such a magnetic resonance apparatus may have a local coil arrangement with a plurality of coil elements for acquiring raw datasets, in particular for receiving raw data, which is contained in the raw datasets, in the form of magnetic resonance signals. These raw datasets and/or raw data may be prescan raw datasets and/or prescan raw data and/or imaging raw datasets and/or imaging raw data.

Furthermore, the magnetic resonance apparatus may have at least one computing unit for generating a plurality of prescan single coil image datasets, for generating a combined prescan image dataset, for generating a plurality of imaging single coil image datasets, and/or for generating the combined imaging image dataset. This computing unit may include one or more processors and/or one or more storage modules.

Furthermore, a computer program product is proposed that includes a computer program and is able to be loaded directly into a memory of a programmable computing unit, in particular a system control unit, of a magnetic resonance apparatus, and has program means, for example, libraries and auxiliary functions in order to carry out a proposed method when the computer program product is executed in the computing unit of the magnetic resonance apparatus. The computer program product may herein include an item of software with a source code to be compiled and linked or which is only to be interpreted, or an executable software code that, for execution, needs only to be loaded into the computing unit.

By the computer program product, the proposed method may advantageously be carried out rapidly, exactly reproducibly, and robustly. The computer program product may be configured to may carry out the proposed method by the computing unit. Therein, the computing unit may have the respective pre-conditions such as a suitable working memory store, a suitable graphics card, or a suitable logic unit so that the method may be carried out efficiently.

The computer program product is stored, for example, on a computer-readable medium or is deposited on a network or server from where it may be loaded into the processor of a local system control unit that may be directly connected to the magnetic resonance apparatus or may be configured as part of the magnetic resonance apparatus. Furthermore, control information of the computer program product may be stored on an electronically readable data carrier. The items of control information of the electronically readable data carrier may be configured such that they carry out a proposed method when the data carrier is used in a computing unit of a magnetic resonance apparatus.

Examples of electronically readable data carriers are a DVD, a magnetic tape or a USB stick, on which electronically readable control information, in particular software, is stored. If this control information is read from the data carrier and is stored in a computing unit of the magnetic resonance apparatus, e.g., all the proposed embodiments of the above-described methods may be carried out.

Further advantages, features, and details of the disclosure become apparent from the description below of embodiments and from the drawings. Parts which correspond to one another are provided with the same reference signs in all the drawings.

### BRIEF DESCRIPTION OF THE DRAWINGS

FIG. 1 depicts an example of a magnetic resonance apparatus.

FIG. 2 depicts an example of a sequence of a method for generating a combined imaging image dataset.

FIG. 3 depicts an example of a spatial signal coverage of a patient by coil elements of a local coil arrangement.

FIG. 4 depicts an example of a matrix for establishing phase values of a combined prescan image dataset.

FIG. 5 depicts an example of resulting magnetic resonance images with and without the use of a proposed method for generating a combined imaging image dataset.

### DETAILED DESCRIPTION

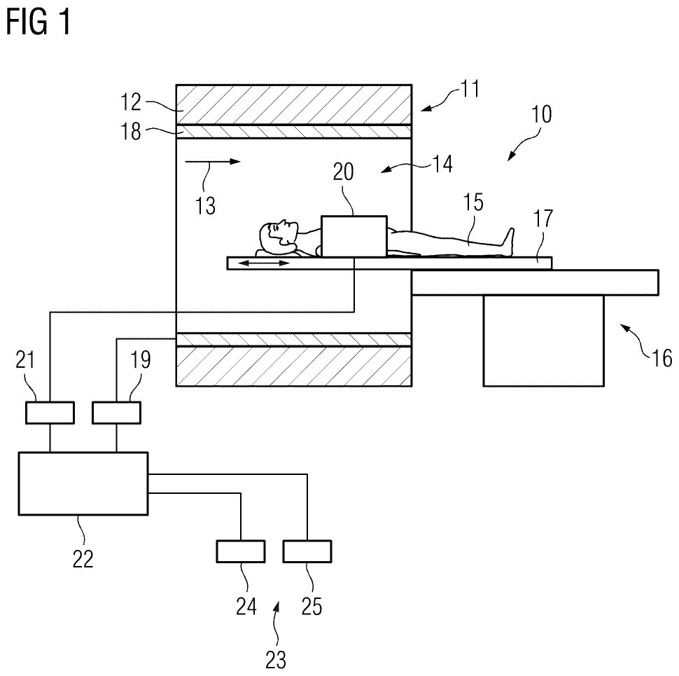

FIG. 1 shows schematically a magnetic resonance apparatus 10. The magnetic resonance apparatus 10 includes a magnet unit 11 which has a main magnet 12 for generating a strong and, in particular, temporally constant main magnet field 13. The main magnet field 13 may have a strength of more than 5 T, for example, 7 T. In addition, the magnetic resonance apparatus 10 includes a patient receiving region 14 for accommodating a patient 15. In the present embodiment, the patient receiving region 14 is cylindrical and is surrounded cylindrically in a circumferential direction by the magnet unit 11. The patient 15 may be moved by a patient positioning apparatus 16 of the magnetic resonance apparatus 10 into the patient receiving region 14. For this purpose, the patient positioning apparatus 16 has a patient table 17 configured to be moved within the patient receiving region 14.

The magnet unit 11 also has a gradient coil unit 18 for generating magnetic field gradients that are used for spatial encoding during an imaging process. The gradient coil unit 18 is controlled by a gradient control unit 19 of the magnetic resonance apparatus 10.

The magnetic resonance apparatus 10 includes no body coil fixedly installed in the magnetic resonance apparatus 10. However, the magnetic resonance apparatus 10, in particular the magnet unit 11 of the magnetic resonance apparatus 10, includes a high frequency antenna unit in the form of a local coil arrangement 20, which, in the present embodiment is arranged in the region of the abdomen of the patient 15. The local coil arrangement 20 may include one or more local coils. For example, the local coil arrangement 20 may include a plurality of local coils positioned adjoining one another on the patient 15.

The local coil arrangement 20 is controlled by a high frequency antenna control unit 21 of the magnetic resonance apparatus 10 and radiates high frequency magnetic resonance sequences, for example, a prescan magnetic resonance sequence or an imaging magnetic resonance sequence into an examination space formed by a patient receiving region 14 of the magnetic resonance apparatus 10. By this, an excitation of atomic nuclei by the main magnet field 13 generated by the main magnet 12 takes place. Through relaxation of the excited atomic nuclei, magnetic resonance signals are generated. With the local coil arrangement 20, the magnetic resonance signals may be received in the form of raw data. The local coil arrangement 20 includes a plurality of coil elements that may separately emit (e.g., excitation signals) and receive (e.g., magnetic resonance signals) HF signals.

For controlling the main magnet 12 and the high frequency antenna control unit, the gradient control unit 19 and magnetic resonance apparatus 10 have a system control unit 22. The system control unit 22 centrally controls the magnetic resonance apparatus 10, for example, the execution of a pre-determined magnetic resonance sequence. In addition, the system control unit 22 includes a computing unit (not shown in further detail) for processing the raw data acquired during the magnetic resonance examination. Furthermore, the magnetic resonance apparatus 10 includes a user interface 23 connected to the system control unit 22. Control information such as imaging parameters and reconstructed magnetic resonance mappings may be displayed on a display unit 24, for example, on at least one monitor of the user interface 23 for medical operating personnel. In addition, the user interface 23 has an input unit 25 by which information and/or parameters may be input by the medical operating personnel during a scanning procedure.

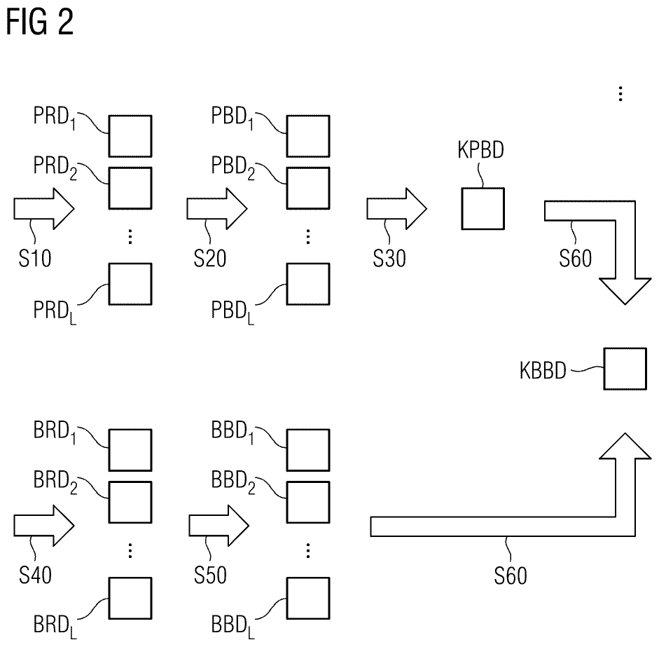

FIG. 2 shows a possible sequence of a method for generating a combined imaging image dataset KBBD on the basis of imaging raw data acquired by the magnetic resonance apparatus 10.

In S10, L prescan raw datasets PRD₁, PRD₂, . . . , PRD_{L} are each acquired with one of the plurality of coil elements, i.e., a first coil element acquires a first prescan raw dataset PRD₁, a second coil element acquires a second prescan raw dataset PRD₂, etc. This means that the local coil arrangement 20 includes at least L coil elements. Not all the coil elements of the local coil arrangement 20 must necessarily be used for the acquisition of raw datasets.

In S20, L prescan single coil image datasets PBD₁, PBD₂, . . . , PBD_{L} are each generated by reconstruction of one of the L prescan raw datasets PRD₁, PRD₂, . . . . PRD_{L}. For example, a first prescan single coil image dataset PBD₁ is generated by way of reconstruction of the first prescan raw dataset PRD₁, a second prescan single coil image dataset PBD₂ is generated by reconstruction of the second prescan raw dataset PRD₂, etc.

In S30, a combined prescan image dataset KPBD is generated by combination of the L prescan single coil image datasets PBD₁, PBD₂, . . . , PBD_{L}. The generation of the combined prescan image dataset KPBD includes an establishment of a plurality of phase values, each of which is associated with an image element of the combined prescan image dataset KPBD. The phase values of the combined prescan image dataset KPBD are established in that the phase values of the combined prescan image dataset KPBD have no singularity, in particular no phase jump.

In S40, L imaging raw datasets BRD₁, BRD₂, . . . , BRD_{L} are acquired, each with one of the plurality of coil elements. The L imaging raw datasets BRD₁, BRD₂, . . . , BRD_{L}, e.g., each have a greater resolution than the corresponding prescan raw datasets PRD₁, PRD₂, . . . , PRD_{L}. For example, the imaging raw dataset BRD₁ has a greater resolution than the corresponding prescan raw dataset PRD₁, etc.

In S50, L imaging single coil image datasets BBD₁, BBD₂, . . . , BBD_{L} are generated, each by reconstruction of one of the L imaging raw datasets BRD₁, BRD₂, . . . , BRD_{L}. For example, a first imaging single coil image dataset BBD₁ is generated by way of reconstruction of the first imaging raw dataset BRD₁, a second imaging single coil image dataset BBD₂ is generated by reconstruction of the second imaging raw dataset BRD₂, etc.

In S60, the combined imaging image dataset KBBD is generated by combining the L imaging single coil image datasets BBD₁, BBD₂, . . . , BBD_{L}, while taking account of the phase values of the combined prescan image dataset KPBD established in S30.

The generation of the datasets in S20, S30, S50, and S60 may be carried out, in particular, with a computing unit which may be part of the system control unit 20.

Still further imaging raw datasets may be acquired and further imaging single coil image datasets generated therefrom and, from these, a further combined imaging image dataset may be generated while taking account of the phase values of the combined prescan image dataset KPBD, while S40, S50 and S60 are repeated. The combined prescan image dataset KPBD may thus advantageously be repeatedly used for a generation of a plurality of combined imaging image datasets.

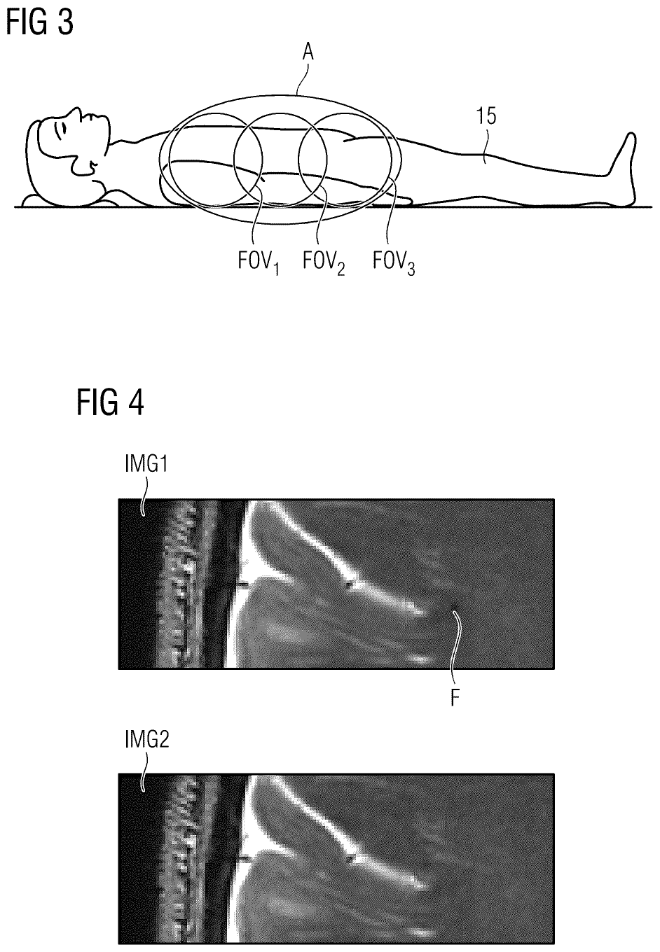

FIG. 3 depicts a patient 15 on whom a local coil arrangement with three coil elements is arranged.

Therein, the fields of view of the three coil elements FOV₁, FOV₂, and FOV₃ are shown. These fields of view are per se not large enough to cover the whole region A to be examined, for example, the region of the abdomen. The coil elements may have no homogeneous reception field. This problem may be solved in that a reference phase is established on the basis of which the relative phase of the individual coil elements may be corrected. The combined prescan image dataset KPBD may describe the phase values established in S30, such a reference phase that is suitable for describing a phase relationship between imaging single coil image data of the L imaging single coil image datasets BBD₁, BBD₂, . . . , BBD_{L}.

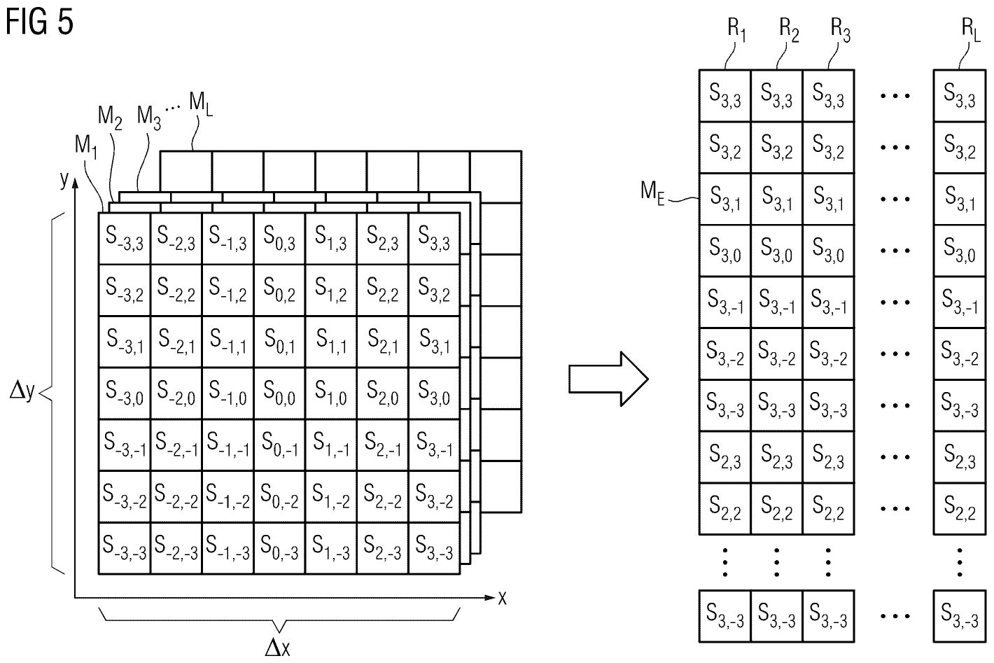

In FIG. 4, on the left, a magnetic resonance image IMG1 is shown, which has been generated without taking account of a reference phase described herein. The magnetic resonance image IMG1 has an artifact F in the form of a signal cancellation. In FIG. 4, on the right, a magnetic resonance image IMG2 is shown, which has been generated while taking account of a reference phase described herein. The magnetic resonance image IMG2 has no such artifact.

In FIG. 5, a matrix M₁ and a matrix M_{E} are shown. Behind the matrix M₁, further matrices M₂, M₃, . . . , M₁ are indicated. Each matrix is therein associated with one of the L prescan single coil image datasets. Each element of the matrix M₁ therein corresponds to an image element with a value S_{x,y} of the associated prescan single coil image dataset. The axes x and y correspond to spatial axes in the image space (that is, not in the k-space). The respective prescan single coil image dataset may have further image elements, i.e., the matrices M₁, M₂, M₃, . . . , M_{L} may each represent only a portion of the respective prescan single coil image dataset. In this portion, there is a central image element with the value S_{0,0}, about which other image elements are arranged. Therein Δx along the x-axis (herein: x=−3, . . . , 0, . . . , 3; that is, N=2·3+1=7 image element coordinates) and Δy along the y-axis (herein: y=−3, . . . , 0, . . . , 3; that is, also M=2·3+1=7 image element coordinates) bound a predetermined region Δx,Δy (which herein also defines the portion shown) about the central image element.

Each value S_{x,y} therein includes, in particular, a phase value. (Apart from the phase value, the value S_{x,y} may include an amplitude value, so that the value S_{x,y} may also have a complex value.) Thus each of the L prescan single coil image datasets PBD₁, PBD₂, . . . , PBD_{L} includes a plurality of phase values, each of which is associated with an image element of the respective prescan single coil image dataset PBD₁, PBD₂, . . . , PBD_{L}.

The combined prescan image dataset KBBD also includes a plurality of phase values, each of which is associated with an image element of the combined prescan image dataset KBBD. An image element of the combined prescan image dataset KBBD (not shown here) corresponds to the image element of the prescan single coil image dataset shown in FIG. 5 with the value S_{0,0}. The establishment of the phase value for this corresponding image element of the combined prescan image dataset KBBD takes place in S30, e.g., while taking account of phase values of the prescan single coil image datasets PBD₁, PBD₂, . . . , PBD_{L}, which (in addition to the corresponding (central) image element) are associated with the image elements that are arranged about the corresponding (central) image element in the predetermined region Δx,Δy.

For example, the phase value for the image element with the image point coordinates x=0, y=0 of the combined prescan image dataset KBBD is established while taking account of the phase values of the image elements of the prescan single coil image datasets PBD₁, PBD₂, . . . , PBD_{L} with the N×M image point coordinates x=−p, . . . , 0, . . . , p where p=3 and y=−q, . . . , 0, . . . , q where q=3; in the example shown, therefore, taking account of the 49 values S_{-3,-3}, . . . , S_{3,3} represented in the matrix M₁, and the corresponding values for M₂, M₃, . . . , M_{L}, i.e., a total of 49×L phase values are taken into account for the establishing of a phase value of the combined prescan image dataset KBBD.

For establishing the phase values of other image elements of the combined prescan image dataset KBBD, the phase values of other image elements are taken into account accordingly. In particular, the predetermined region from image element to image element is displaced. For image elements of the combined prescan image dataset KBBD that are close to the boundary, the predetermined region in the prescan single coil image datasets PBD₁, PBD₂, . . . , PBD_{L} is, where necessary, adapted so that the image element corresponding to the image element close to the boundary is no longer situated centrally in the predetermined region.

For establishing a phase value associated with an image element, a matrix M_{E} is generated. The matrix M_{E} herein has, for each prescan single coil image dataset PBD₁, PBD₂, . . . , PBD_{L}, a column R₁, R₂, R₃, . . . , R_{L} with the respective phase values S_{-3,-3}, . . . , S_{3,3}, so that the matrix M_{E} includes the phase values of all the prescan single coil image datasets PBD₁, PBD₂, . . . , PBD_{L}, which are associated with the image elements that are situated in the predetermined region Δx,Δy. Eigenvalues of the generated matrix M_{E} are established, wherein the phase value of the combined prescan image dataset KPBD associated with the image element is stipulated on the basis of the maximum established eigenvalue.

Finally, the method described above in detail and the magnetic resonance apparatus disclosed are merely exemplary embodiments which may be modified by a person skilled in the art in a wide variety of ways without departing from the scope of the disclosure. Furthermore, the use of the indefinite article “a” or “an” does not preclude the possibility that the relevant features may also be present more than once. Similarly, the expression “unit” does not preclude the relevant components including a plurality of cooperating sub-components which may also be spatially distributed, if relevant.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for generating a combined imaging image dataset based on raw data acquired by a magnetic resonance apparatus, wherein the magnetic resonance apparatus comprises a local coil arrangement having a plurality of coil elements, the method comprising:
acquiring a plurality of prescan raw datasets, each prescan raw dataset being acquired with one coil element of the plurality of coil elements;
generating a plurality of prescan single coil image datasets by reconstructing each individual prescan raw dataset of the plurality of prescan raw datasets;
generating a combined prescan image dataset by combining the plurality of prescan single coil image datasets, wherein the generating of the combined prescan image dataset comprises an establishment of a plurality of phase values, each phase value of which is associated with an image element of the combined prescan image dataset, and wherein the plurality of phase values of the combined prescan image dataset have no phase jump;
acquiring a plurality of imaging raw datasets, each imaging raw dataset being acquired with one coil element of the plurality of coil elements;
generating a plurality of imaging single coil image datasets by reconstructing each individual imaging raw dataset; and
generating the combined imaging image dataset by combining the plurality of imaging single coil image datasets while taking account of the plurality of phase values of the combined prescan image dataset.

2. The method of claim 1, wherein the plurality of phase values of the combined prescan image dataset is established in that the plurality of phase values of the combined prescan image dataset has no phase jump of 360° and/or 2π.

3. The method of claim 1, wherein the combined prescan image dataset describes a reference phase suitable for describing a phase relationship between imaging single coil image data of the plurality of imaging single coil image datasets.

4. The method of claim 1, wherein the local coil arrangement comprises one or more local coils.

5. The method of claim 1, wherein each prescan raw dataset of the plurality of prescan raw datasets has a lower resolution than the corresponding imaging raw dataset of the plurality of imaging raw datasets.

6. The method of claim 1, further comprising:
acquiring a plurality of further imaging raw datasets, each further imaging raw dataset being acquired with one coil element of the plurality of coil elements;
generating a plurality of further imaging single coil image datasets by reconstructing each individual further imaging raw dataset of the plurality of further imaging raw datasets; and
generating a further combined imaging image dataset by combining the plurality of further imaging single coil image datasets while taking account of the plurality of phase values of the combined prescan image dataset.

7. The method of claim 1, wherein each prescan single coil image dataset of the plurality of prescan single coil image datasets comprises a plurality of phase values that are each associated with one image element of the respective prescan single coil image dataset, and
wherein the phase values of the plurality of phase values of the combined prescan image dataset, which are each associated with one image element of the combined prescan image dataset, are established while taking account of a plurality of phase values of the plurality of prescan single coil image datasets that are associated with image elements arranged in a predetermined region about the respective image element.

8. The method of claim 7, wherein for establishing a phase value associated with an image element, a matrix is generated that comprises the phase values of all the prescan single coil image datasets associated with the image elements situated in the predetermined region,
wherein eigenvalues of the generated matrix are established, and
wherein the phase value of the combined prescan image dataset associated with the image element is stipulated based on a maximum established eigenvalue.

9. The method of claim 1, wherein, based on the plurality of phase values of the combined prescan image dataset, coil combination weights are established, and
wherein the plurality of imaging single coil image datasets are combined to the combined imaging image dataset using on the coil combination weights.

10. The method of claim 1, wherein the magnetic resonance apparatus has a main magnet field of more than 5 T.

11. The method of claim 1, wherein the magnetic resonance apparatus has no body coil fixedly installed in the magnetic resonance apparatus.

12. A magnetic resonance apparatus comprising:
a local coil arrangement having a plurality of coil elements,
wherein the magnetic resonance apparatus is configured to:
acquire a plurality of prescan raw datasets, each prescan raw dataset being acquired with one coil element of the plurality of coil elements of the local coil arrangement;
generate a plurality of prescan single coil image datasets by reconstructing each individual prescan raw dataset of the plurality of prescan raw datasets;
generate a combined prescan image dataset by combining the plurality of prescan single coil image datasets, wherein the generating of the combined prescan image dataset comprises an establishment of a plurality of phase values, each phase value of which is associated with an image element of the combined prescan image dataset, and wherein the plurality of phase values of the combined prescan image dataset have no phase jump;
acquire a plurality of imaging raw datasets, each imaging raw dataset being acquired with one coil element of the plurality of coil elements;
generate a plurality of imaging single coil image datasets by reconstructing each individual imaging raw dataset; and
generate the combined imaging image dataset by combining the plurality of imaging single coil image datasets while taking account of the phase values of the combined prescan image dataset.

13. A computer program product comprising a computer program, wherein the computer program is configured to be loaded directly into a memory of a programmable computing unit of a magnetic resonance apparatus, wherein the computer program, when executed by the programmable computing unit, is configured to cause the magnetic resonance apparatus to:
acquire a plurality of prescan raw datasets, each prescan raw dataset being acquired with one coil element of a plurality of coil elements of a local coil arrangement of the magnetic resonance apparatus;
generate a plurality of prescan single coil image datasets by reconstructing each individual prescan raw dataset of the plurality of prescan raw datasets;
generate a combined prescan image dataset by combining the plurality of prescan single coil image datasets, wherein the generating of the combined prescan image dataset comprises an establishment of a plurality of phase values, each phase value of which is associated with an image element of the combined prescan image dataset, and wherein the plurality of phase values of the combined prescan image dataset have no phase jump;
acquire a plurality of imaging raw datasets, each imaging raw dataset being acquired with one coil element of the plurality of coil elements;
generate a plurality of imaging single coil image datasets by reconstructing each individual imaging raw dataset; and
generate the combined imaging image dataset by combining the plurality of imaging single coil image datasets while taking account of the phase values of the combined prescan image dataset.
